(19) United States
(12) Patent Application Publication
(10) Pub. No.: US 2009/0219485 A1
(43) Pub. Date: Sep. 3, 2009

(54) Ocular wavefront system

(76) Inventor: Edwin J. Sarver, Carbondale, IL (US)

Correspondence Address: MCHALE & SLAVIN, P.A., 2855 PGA BLVD, PALM BEACH GARDENS, FL, 33410, US

(21) Appl. No.: 12/393,761

(22) Filed: Feb. 26, 2009

Related U.S. Application Data
(60) Provisional application No. 61/032,646, filed on Feb. 29, 2008.

(51) Int. Cl. A61B 3/10

(52) U.S. Cl. 351/221; 351/246

(57) ABSTRACT

The present invention generally relates to systems for measuring aberrations of the eye and more specifically to an ocular wavefront system adapted to measure aberrations of the eye for vision correction.

### CROSS-REFERENCE TO RELATED APPLICATIONS

This application claims benefit of the filing date of U. S. Provisional Patent Application No. 61/032,646, filed on Feb. 29, 2008, the contents of which are herein incorporated by reference.

### FIELD OF INVENTION

The present invention generally relates to systems for measuring aberrations of the eye and more specifically to an ocular wavefront system adapted to measure aberrations of the eye for vision correction.

### BACKGROUND INFORMATION

In the course of daily life, one typically regards objects located at different distances from the eye. To selectively focus on such objects, the focal length of the eye's lens must change. In a healthy eye, this is achieved through the contraction of a ciliary muscle that is mechanically coupled to the lens. To the extent that the ciliary muscle contracts, it deforms the lens. This deformation changes the focal length of the lens. By selectively deforming the lens in this manner, it becomes possible to focus on objects that are at different distances from the eye. This process of selectively focusing on objects at different distances is referred to as “accommodation.”

As a person ages, the lens loses plasticity. As a result, it becomes increasingly difficult to deform the lens sufficiently to focus on objects at different distances. This condition is known as presbyopia. Refractive errors caused by such conditions as hyperopia, myopia, as well as aberrations due to irregularities in the eye (e.g., in the cornea or in the natural crystalline lens) can also degrade one's ability to focus on an, object. To compensate for this loss of function, it is useful to provide different optical corrections for focusing on objects at different distances.

One approach to applying different optical corrections is to carry different pairs of glasses and to swap glasses as the need arises. For example, one might carry reading glasses for reading and a separate pair of distance glasses for driving.

In another approach, bifocal lenses assist accommodation by integrating two different optical corrections onto the same lens. The lower part of the lens is ground to provide a correction suitable for reading or other close-up work while the remainder of the lens is ground to provide a correction for distance vision. To regard an object, a wearer of a bifocal lens need only maneuver the head so that rays extending between the object-of-regard and the pupil pass through that portion of the bifocal lens having an optical correction appropriate for the range to that object.

Laser eye surgery techniques for improving focusing ability involve laser ablation of a portion of the eye. In Photorefractive Keratectomy (PRK) surgery, a surgeon uses an excimer laser to remove tissue from the surface of the cornea. In Laser-Assisted In Situ Keratomileusis (LASIK) surgery or Laser Epithelial Keratomileusis (LASEK) surgery, a surgeon removes tissue under the surface of the cornea by lifting a portion (a “flap”) of the cornea. Tissue is selectively removed to reshape the cornea so that less deformation of the lens is necessary for accommodation. Customized laser eye surgery based on measurements of a subject's eye can also compensate for some wavefront aberrations. During laser eye surgery, the cornea is reshaped to improve vision for a single distance of regard. Vision at other distances may remain degraded. For example, even after laser eye surgery, a subject may still need to use glasses to correct far vision. Therefore, there is a need in the art for a low-cost ocular wavefront system suitable for measuring the aberrations of the eye so that a clinician can use the information to evaluate of treat a patient's vision.

### SUMMARY OF THE INVENTION

Thus, the present invention overcomes the disadvantages of the prior art, described above, by providing a low-cost ocular wavefront system that measures the aberrations of the eye so that a clinician can use the information to evaluate of treat a patient's vision.

Accordingly, it is an objective of the present invention to provide an ocular wavefront system that measures the aberrations of the eye.

It is a further objective of the instant invention to provide a method for providing multi-focal visual correction.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 is a schematic illustrating one embodiment of the ocular wavefront system of the instant invention;

FIG. 2 is a schematic illustrating the sensor path details of the system illustrated in FIG. 1;

FIG. 3 is a front view illustrating a Hartman screen suitable for use with the instant invention;

FIG. 4 illustrates field of view at the eye's pupil.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

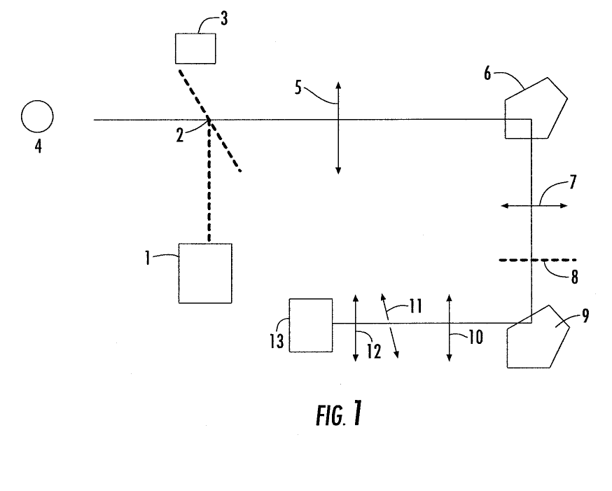

Referring generally to FIG. 1, an 830 nm super luminescent diode (SLD) 1 emits a beam which is directed toward pellicle beam splitter 2. Some of the light from the SLD passes through the beam splitter and is absorbed and redirected by a beam dump 3. The remainder of the light from the SLD is directed into the eye 4. This light forms a diffuse source at the back of the eye. The SLD is preferably always on, and its output power is maintained at a level that is safe for continuous viewing. The wavefront exiting the patient's eye at the entrance pupil (about 3 mm inside the eye) passes back through the pellicle beam splitter and is refracted by a first lens 5 (L1), redirected by penta prism 6, and refracted by a second lens 7 (L2) to make the wavefront available to the Hartman screen 8. In a most preferred embodiment, L1 5 and L2 7 are equal focal length lenses. A penta prism 6 is utilized to redirect the light from the horizontal optical axis to the vertical optical axis so that the beam is rotated 90 degrees even if the penta prism is not exactly aligned and helps reduce the overall size of the system. The Hartmann screen (HS) aperture array serves to divide the incident wavefront into an array of spots that fall onto the sensor plane. The focal plane of the HS sensor is imaged on to a charge coupled device sensor (CCD sensor) 13 via a third lens 10 (L3), an aperture 11, and fourth lens 12 (L4). As noted the penta prism 9 is used to redirect light through a 90 degree bend with the properties that it is insensitive to rotation errors and helps reduce the physical size of the system. To provide fine alignment of the system, the SLD 1, L1 5, and CCD sensor 13 are mounted on adjustable fixtures. In operation, the patient observes the SLD 1 as a fixation source. This ensures that the SLD 1 light is properly positioned on the subject's retina.

Referring to FIG. 2, the sensor path (straightened for ease of explanation) is illustrated. In the preferred embodiment, the focal lengths of lens 1 and lens 2 equal 100 mm and have 1:1 imaging at the hue saturation (HS) plane. This means d1=200 mm. For example, under these conditions, the following formula may be utilized.

2f1−d0=d2 (1)

Choosing d0=150 mm to give at least 50 mm working distance in front of the system gives d2=50 mm. The distance d3 is set to a focal plane of the Hartmann screen. This focal plane distance is calculated using a well known relation from diffraction theory. This relation is given in equation (2) below.

$$f \approx \frac{{R}^{2}}{λ} \qquad ( 2 )$$

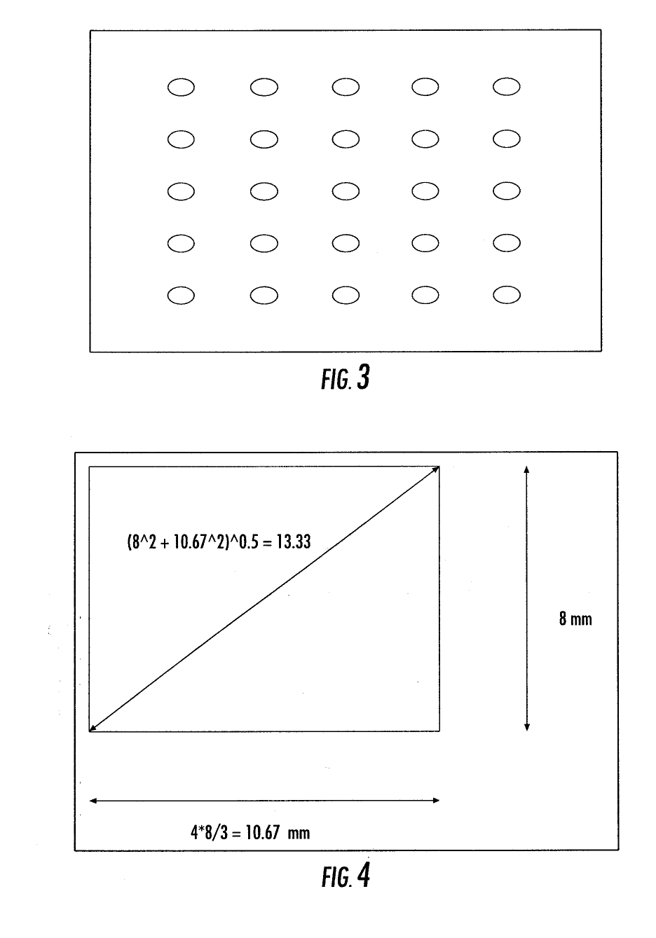

Equation (2) shows the focal distance for the aperture used as a lens. For a Hartmann screen with apertures spaced 0.25 mm apart having an aperture radius R=0.125 mm the resulting focal distance is about 4.7 mm. The Hartmann screen is illustrated in FIG. 3. To compute the third lens and the fourth lens the vertical field of view at the pupil's entrance plane VFOV and the height of the CCD sensor H in is needed, preferably in mm. For example, if VFOV=8 mm and H=3.6 mm (1/3″ sensor). The required magnification is computed as 0.45 from:

$$mag = \frac{H}{VFOV} = 0.45 \qquad ( 3 )$$

If L3 is chosen to be 100 mm and L4 is chosen to be 40 mm since they are commonly available lens focal lengths, selecting both L3 and L4 as achromats provides a high quality imaging system with only two lenses. The resulting magnification is 0.4 which is close enough to our desired magnification of 0.45. Thus, d4 is 100 mm and d5 is 40 mm.

For an f# =5 the aperture between lens 3 and lens 4 (shown as item 11 in FIG. 1) should be:

$$\begin{matrix}D = \frac{xi}{f \#} \\ = \frac{40}{5} \\ = 8\end{matrix} \qquad ( 4 )$$

To compute the required clear aperture for lens 1 and lens 2 for no vignetting at the CCD proceed as follows. The vertical and horizontal FOV at the eye's pupil is illustrated with reference to FIG. 4.

The ray height at the edge will be 13.33/2=6.67 mm. For a 10 diopter diverging wavefront at the pupil, the ray slope v may be calculated with the following formula (5):

$$\begin{matrix}V = \frac{D}{1000} \\ = \frac{10}{1000} \\ = 0.01\end{matrix} \qquad ( 5 )$$

Now, system matrices are used for the S1=distance between eye's pupil and the first lens and S2 =S1+refraction at L1 and the distance between L1 and L2, to determine the ray height at the lenses L1 and L2. The values r, S1 and S2 are determined by the following matrices (6)

$$r = [ \begin{matrix}6.67 \\ 0.01\end{matrix} ] \\ S 1 = [ \begin{matrix}1 & 150 \\ 0 & 1\end{matrix} ] \\ S 2 = [ \begin{matrix}- 1 & 50 \\ - 0.01 & - 0.5\end{matrix} ] \qquad ( 6 )$$

The matrix multiplication yields: 8.17 at L1 and −6.17 at L2. Thus, 20 mm diameter optics at L1 and L2 are sufficient.

Traditional processing of the captured image follows the steps of:

1. Find the centroids of the spots arrays.

2. Find the deviation of each spot in x and y relative to a reference image for a plane wave.

3. Calculate the wavefront derivative in x and y using the deviations found in step 2.

4. Fit the wavefront derivatives to a Zernike polynomial to yield the desired ocular wavefront.

Instead of the traditional processing, in our preferred embodiment phase recovery techniques are used to obtain the wavefront slopes. Explaining the operation of the Fourier transform phase recovery technique first facilitates the explanation of the spatial demodulation technique to follow. Thus, we begin with a discussion of the Fourier transform technique. It is convenient to represent the irradiance distribution of the HS spot image for an incident plane wave as the infinite cosine function product g₀(x,y) times a spatial domain aperture function that has a value of 1 inside the pupil and 0 outside. The function g₀(x,y) is given by the following equation (7).

$${g}_{0} ( x , y ) = [ \frac{1}{2} + \frac{1}{2} cos ( \frac{2 π}{{p}_{x}} x ) ] \times [ \frac{1}{2} + \frac{1}{2} cos ( \frac{2 π}{{p}_{y}} y ) ] \qquad ( 7 )$$

In this equation, px and py are the period of the HS array spacing in the x and y directions. The corresponding Fourier transform G₀(u,v) is a two-dimensional array of weighted delta functions:

$${G}_{0} ( u , v ) = \frac{1}{4} δ ( 0 , 0 ) + \frac{1}{8} [ δ ( u - \frac{1}{{p}_{x}} , v ) + δ ( u + \frac{1}{{p}_{y}} , v ) ] … \qquad ( 8 )$$

For a sufficiently wide aperture the Fourier transform of the aperture function times the function g₀ is only slightly different from the simple delta functions of equation (8). When the local slope of the incident wavefront is not zero, the irradiance distribution can be thought of as being warped by a coordinate transformation. The value of delY (and similar for delX) is given by

$$del Y = - \frac{d W ( y )}{d y} \times f \qquad ( 9 )$$

The irradiance distribution for the aberrated incident wavefront is denoted g1(x,y) and is given by

$$\begin{matrix}{g}_{1} ( x , y ) = {g}_{0} [ x + A ( x , y ) , y + B ( x , y ) ] \\ = [ \frac{1}{2} + \frac{1}{2} cos ( \begin{matrix}\frac{2 π}{{p}_{x}} \\ ( x + A ( x , y ) )\end{matrix} ) ] \times [ \frac{1}{2} + \frac{1}{2} cos ( \begin{matrix}\frac{2 π}{{p}_{y}} \\ ( y + B ( x , y ) )\end{matrix} ) ]\end{matrix} \qquad ( 10 )$$

where A(x,y) and B (x,y) are proportional to the partial derivatives of the wavefront with respect to x and y. Now, writing the cosine functions as a sum of complex exponentials and taking the Fourier transform, we see that the function A(x,y) appears as the argument of a complex exponential shifted to the frequency 1/px along the u axis in the Fourier domain. Likewise, the function B(x,y) appears as the argument of a complex exponential shifted to the frequency 1/py along the v axis in the Fourier domain. Note that there are two regions that must be processed: one for wavefront derivatives with respect to x and the other for wavefront derivatives with respect to y. Now the top level steps used in the Fourier transform technique can be enumerated:

1. Compute the Fourier transform of the HS image.

2. Isolate the region of interest in the Fourier domain and shift the center of the region of interest to the origin.

3. Compute the inverse Fourier transform and compute the complex angle to yield the wrapped phase.

Two additional steps are needed to complete the process.

4. Unwrap the phase and normalize to yield the wavefront gradient; and

5. Reconstruct the wavefront from the gradients.

In step 4, we need to unwrap the phase from the arrays (one for dW/dx and the other for dW/dy) computed in step 3 and then normalize the arrays to account for the micro lens focal length. Note that the normalization must be performed after the unwrapping. When there are no jump discontinuities (called residues) in the phase arrays, the unwrapping can be accomplished with a simple and fast algorithm. In this case, the unwrapping algorithm (in one dimension with U=unwrapped and W=wrapped) can be described as:

### Set U(0)=W(0)

For n=1 to the end of the array do the following

D=W(n)−W(n−1)

If D<−PI then D=D+2Pi

If D>Pi then D=D−2Pi

U(n)=U(n−1)+D

This algorithm can be applied down the center of the array and then to the left and right to the edges of the arrays. Where there are phase discontinuities, more elaborate phase unwrapping techniques must be employed as are known to those skilled in the art. After unwrapping and normalizing to obtain the wavefront slopes in the x and y directions, the wavefront is constructed by fitting to a Zernike or Fourier expansion.

### Spatial Demodulation

The previous processing using Fourier transforms can also be accomplished entirely in the spatial domain. In this method, the spots image is multiplied by a complex exponential to shift the desired neighborhood to the origin in the frequency domain. This operation can be described by the Fourier transform modulation relation shown in equation (11).

$${e}^{- j2π ax} f ( x ) {}_{FT} F ( s + a ) \qquad ( 11 )$$

One complex exponential is used to obtain the neighborhood for the wavefront slopes with respect to x and another is used to obtain the neighborhood for the wavefront slopes with respect to y. A low-pass filter is then used to isolate the desired frequency band. This produces the wrapped phase arrays as we obtained in the Fourier method. The remaining steps of unwrapping the phase and reconstruction the wavefront are the same as for the Fourier method. To be practical, the low-pass filter must be very efficient since it is computed in the spatial domain using convolution. One example of such a computationally efficient low-pass filter is the box filter in which all coefficients of the impulse response are equal. When implemented as a sliding sum, the filter is computed using only 2 adds and 2 subtracts per output sample. We use two passes of this filter to provide a triangle shaped impulse response which has much less energy in the side lobes of the filter's frequency response compared to the box filter. The main steps in the spatial demodulation technique are:

1. Multiply the spots image by a complex exponential.

2. Isolate the region of interest by applying a low-pass filter.

3. Unwrap the phase and normalize to yield the wavefront gradient.

4. Reconstruct the wavefront from the gradients.

### Combination of Reconstruction Methods

To improve accuracy of the reconstructed ocular wavefront, we reconstruct the wavefront first using one method (for example the traditional method described above) and then another method (for example the spatial demodulation method described above). By averaging the two Zernike expansions representing the wavefronts, the accuracy of the measurements is increased. By evaluating the difference in the measurements provided by the two methods, we can determine how well the wavefront was likely obtained. That is, if the differences between the two methods is small, the resulting average is likely a good representation of the ocular wavefront. If the differences are large, one or more wavefront is likely in error and the ocular exam should be repeated to obtain a better (more accurate) measurement.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. Any compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. The product comprising any feature described, either individually or in combination with any feature, in any configuration.

2. The process of measuring aberrations in the eye comprising any step described, either individually or in any combination with any step, in any order.
